(19) United States
(12) Patent Application Publication
(10) Pub. No.: US 2020/0331983 A1
(43) Pub. Date: Oct. 22, 2020

(54) NARCOLEPSY-SPECIFIC MARKERS

(71) Applicant: University of Helsinki, Helsingin Yliopisto (FI)

(72) Inventors: Antti Vaheri, Helsingin Yliopisto (FI); Olli Vapalahti, Helsingin Yliopisto (FI); Markku Partinen, Helsingin Yliopisto (FI); Kaia Palm, Tallinn (EE); Toomas Neuman, Tallinn (EE); Arno Pihlak, Tallinn (EE)

(21) Appl. No.: 16/303,734

(22) PCT Filed: May 26, 2017
(86) PCT No.: PCT/FI2017/050396

§ 371 Date: Nov. 21, 2018

(30) Foreign Application Priority Data
May 26, 2016 (FI) 20165442

(51) Int. Cl. C07K 14/72; G01N 33/564; G01N 33/88; C07K 16/26

(52) U.S. Cl. CPC C07K 14/72; G01N 33/564; G01N 2800/2864; C07K 16/26; G01N 33/88

(57) ABSTRACT

The disclosure relates to novel epitopes specific for narcolepsy and their uses in methods for evaluating a subject for narcolepsy, in medical and diagnostic devices and in therapy.

Incorporated herein by reference in its entirety is a Sequence Listing, comprising SEQ ID NO: 1 to SEQ ID NO: 13. The Sequence Listing is a file submitted herewith, the Sequence Listing being in a computer-readable ACSII file named 31379WO_narcolepsy_rev_ST25 created on 7 Nov. 2018 and of 5 KB in size.

### FIELD

The present disclosure is related to the field of biological markers and for diagnosing narcolepsy and for treatment and treatment monitoring of narcolepsy.

### BACKGROUND

Narcolepsy is a chronic neurological disease characterized by irresistible daytime sleepiness and disturbed nocturnal sleep. A major hallmark of narcolepsy is the disturbance of hypothalamic function. Narcolepsy is considered to be an autoimmune disease as there is a strong association with HLA DQB1*06:02 allele. Polymorphisms in loci for T-cell receptor-α and P2RY11 have been associated with narcolepsy. Recently, TRIB-2 protein was identified as a putative target for autoantibodies in narcolepsy. It was detected in 14% of narcoleptic patients compared with 5% in healthy controls. In addition, environmental triggers likely play a role. Streptococcal and influenza infections may be contributing to narcolepsy development in individuals with genetic risk of the disease.

The prevalence of narcolepsy varies from 3 to 5/10 000 in the Caucasian general population. There are also some reports of narcolepsy symptoms associated to organic lesions such as mid-brain tumors, pontine gliomas, hypothalamic gliosis, and Down syndrome.

During 2009, 6 million individuals in Sweden and 2.8 million individuals in Finland were vaccinated with the influenza vaccine Pandemrix (Glaxo Smith Kline) to limit the spread of the pandemic influenza A H1N1v strain. Notably, at the time of release to the market in October 2009, the safety experience of Pandemrix was deemed to be limited. In the autumn of 2010 an unexpectedly large number of reports on narcolepsy in adolescents and children was noted by the Medical Products Agency in Sweden (as in Finland). Subsequent epidemiological studies in Sweden and Finland reported several-fold increased risks of narcolepsy in children and adolescents. However, narcolepsy poses a global health and economic burden as currently ca 330 000 people are diagnosed yearly with narcolepsy, and altogether there are more than 3 million people suffering from this disorder worldwide.

A large population study confirmed an increased risk for a diagnosis of narcolepsy in individuals 20 years of age and observed a trend towards an increased risk also amongst young adults between 21 and 30 years. Recent studies found autoantibodies against the protein TRIB2, but this finding was replicated only in a subset of recent-onset patients. Despite numerous efforts, however, the definitive proof for autoimmunity, the presence of autoantibodies or T-cell responses directed towards hypocretin (orexin) cell antigens, is lacking.

Current diagnosis of narcolepsy is based on clinical data and poly-somnographic studies. These studies are expensive and require specialized facilities with costly equipment. Overall, diagnosis comes with years of delay due to the unspecificity of the symptoms, high economic costs and low reliability of the diagnostic methods.

There is therefore an obvious need to provide a diagnostic method for narcolepsy that overcomes above problems. In order to obtain wide acceptance, the method should preferably be not only reliable and robust, but also easy to perform without excessive inconveniences for the subject.

### SUMMARY

The aspects of the disclosed embodiments provide a method based on the detection of at least one narcolepsy-specific antibody binding to a presently identified epitope. The method is useful since it provides a new easy, safe, cost-effective and fast detection tool for the diagnosis of narcolepsy.

According to the first aspect is provided a method of evaluating a subject for narcolepsy comprising
a. obtaining a biological sample from the subject;
b. assaying the biological sample for an antibody specific for at least one epitope specific for narcolepsy to obtain an assay result for the biological sample;
c. comparing the assay result for the biological sample with an assay result for a reference sample or with a predetermined concentration level of the antibody; and
d. concluding that said subject is at increased risk of having, or has acquired, narcolepsy if the assay result of the biological sample is higher than the assay result of said reference sample, or is different by a predetermined concentration level.

In an embodiment the method is carried out in the sequence a, b, c and d.

In another embodiment the assay for the biological sample and for the reference sample, or which is used to obtain a predetermined concentration level, is carried out using the same assay.

According to a second aspect is provided an epitope specific for narcolepsy comprising the sequence [K/R/H]-X-X-X-[A/V/L/I/P/W/F/M]-X-X-[D/E], SEQ ID NO: 1. or SEQ ID NO: 2 or SEQ ID NO: 3.

According to a third aspect is provided glass, plastic, carbon, polymer based, metallic or silicon plate, slide, chip or cartridge comprising at least one epitope of the second aspect attached on it directly or by using a binding partner or a linker.

According to an aspect is provided glass, plastic, carbon, polymer based, metallic or silicon plate, slide, chip or cartridge comprising at least one epitope of DP1 attached on it directly or by using a binding partner or a linker.

According to a fourth aspect is provided a kit comprising the glass, plastic, carbon, polymer based, metallic or silicon plate, slide, chip or cartridge of the third aspect, optionally a reference sample, and instructions for using it to assay a biological sample derived from a subject, preferably a human, to determine if said subject has or is at risk of developing narcolepsy.

According to a fifth aspect is provided a use of the epitope of the second aspect as an immunoadsorbent for plasmapheresis and/or cytopheresis.

According to a fifth aspect is provided a use of the epitope of DP1 as an immunoadsorbent for plasmapheresis and/or cytopheresis.

According to an aspect is provided a use of the epitope of the second aspect in the diagnosis of narcolepsy.

According to an aspect is provided a use of DP1 or an epitope of DP1 in the diagnosis of narcolepsy.

According to a sixth aspect is provided an antibody specific for the epitope of the second aspect.

According to a seventh aspect is provided a peptide comprising the sequence of SEQ ID NO: 1, SEQ ID NO: 2 or SEQ ID NO: 3, or the present epitope specific for narcolepsy for treating narcolepsy. The peptide may achieve the therapeutic function by allowing formation of a peptide-Ab complex, which may be removed from the body naturally. In an embodiment the peptide is a recombinant protein. In another embodiment the peptide comprises a label or a tag, such as an affinity tag or a detection tag.

According to another aspect is provided a composition comprising a peptide comprising the sequence of SEQ ID NO: 1, SEQ ID NO: 2 or SEQ ID NO: 3, or the present epitope specific for narcolepsy, and optionally at least one of the following: a carrier, a buffer, and a preservative. In an embodiment the composition is in a dried, such as lyophilized, form. In another embodiment it is in a wet form, such as in an aqueous form. In another embodiment the composition is a pharmaceutical composition comprising a therapeutically active amount of the peptide.

According to another aspect is provided a peptide comprising an epitope-mimicking peptide which mimics the narcolepsy-specific biomarker of SEQ ID NO: 1, SEQ ID NO: 2 or SEQ ID NO: 3, or the present epitope specific for narcolepsy, for treating narcolepsy. The mimicking may be as a result of structural changes in the peptide giving it similar or identical functions or actions compared to the naturally occurring epitope. However, the changes may result into better stability or efficacy in diagnostic tests or therapy. The mimicking peptides may mimic chemical, physical or functional property or properties of the original epitope or a part of the epitope.

According to an eighth aspect is provided a method of targeted narcolepsy-specific antibody removal from a biological fluid of a subject in need thereof, the method comprising:

contacting a sample of biological fluid, optionally taken from the subject, with a surface or particle to which is immobilized an epitope specific for narcolepsy, which selectively binds to the narcolepsy-specific antibody suspected of being in the sample of biological fluid; and

wherein the narcolepsy-specific epitope comprises a peptide having a sequence selected from SEQ ID NO: 1, SEQ ID NO: 2 and SEQ ID NO: 3, or the present epitope specific for narcolepsy.

In an embodiment of the eighth aspect the method is an in vitro method.

In a ninth aspect is provided an apheresis device comprising a peptide comprising a sequence selected from SEQ ID NO: 1, SEQ ID NO: 2 and SEQ ID NO: 3, or the present epitope specific for narcolepsy.

In an embodiment the apheresis device is a plasmapheresis device.

The diagnostic use of the test and the method is important in the initial differential diagnosis, in particular in the study of suspected or possible autoimmune mediated neurological and psychiatric diseases, such as chronic fatigue syndrome, Parkinson's disease, Kleine-Levin syndrome, narcolepsy and related sleep disorders, schizophrenia and Alzheimer's disease. They may also be useful means of screening for the study of narcolepsy in risk populations.

They may also be important as a means of screening for studying populations or groups requiring treatment of narcolepsy.

The present invention also provides a method for treating narcolepsy comprising the use of serologic antigen mimicking peptides to eliminate the narcolepsy-specific antibodies by removing them or specific B-cells in plasmapheresis or cytopheresis using specific immunoadsorbents. According to another aspect is provided a method of treating narcolepsy comprising the extracorporeal removal of disease-associated antigen response (antibodies) from a subject's plasma and returning the plasma to the subject.

### BRIEF DESCRIPTION OF THE FIGURES

FIG. 1 shows results that seroreactivity against selected set of peptides distinguishing narcolepsy samples is significantly elevated in the narcolepsy group compared to the H1N1-infected and H1N1-vaccinated groups. Box-and-whisker plot showing Log-transformed values of total IgG response to Pandemrix-induced narcolepsy-specific peptide antigens (1644 peptides compiling the R...P..D motif) in samples from H1N1-infected and H1N1-vaccinated or from patients with narcolepsy. The logarithm-transformed IgG response values for the majority (i.e., the “box” part of the box-whisker plot) of narcolepsy samples (n=16) were greater than that of H1N1-infected and Pandemrix-vaccinated samples (n=16+16). The minimum, first quartile, median, third quartile, and maximum of the logarithm-transformed peptide antigen sum values were 1.25, 1.47, 1.66, 1.78.

FIG. 2 illustrates evaluation of the predominant epitope carrying peptide with the RVLAPALDSWGT sequence for antibodies in the serum samples of patients diseased with narcolepsy. Evaluation of binding activity of screened RVLAPALDSWGT peptide by phage dot blot ELISA. Phages dotted at 2 different dilutions (1:2, 1:4) on nitrocellulose membrane were incubated with 4 different study sera diluted at 1:200 (NRC-S-0, NRC-S-59, NRC-S-25 and NRC-S-71). Upper part of the figure illustrates signal stemming of bound human IgG (visualized by Cy5-conjugated Affinipure Goat anti-Human IgG (H+L)) and lower part the staining of phages with mouse anti-M13 pVIII monoclonal antibody (visualized by Alexa fluor 488 F(ab′)₂ fragment of Goat anti-mouse IgG (H+L). Human IgG (50 ng/μl) was used as positive control (marked with number 3).

FIGS. 3A and 3B: RxxxPxxD motif carrying peptides discriminate between Pdmx-HD (n=16) and Pdmx-NC (n=16) cohorts in ELISPOT assay. Relative units are calculated as fold change difference between ELISPOT signals detected by RxxxPxxD_short and_long and corrected by signals detected with RxxxPxxD_neg in the same individual samples and mean values calculated for both groups. The results show that the non-variable residues of the RxxxPxxD motif are important for discriminating between the studied cohorts. Pdmx-NC, Pandemrix-induced narcolepsy; Pdmx-HD, Pandemrix-vaccinated-NC diseased.

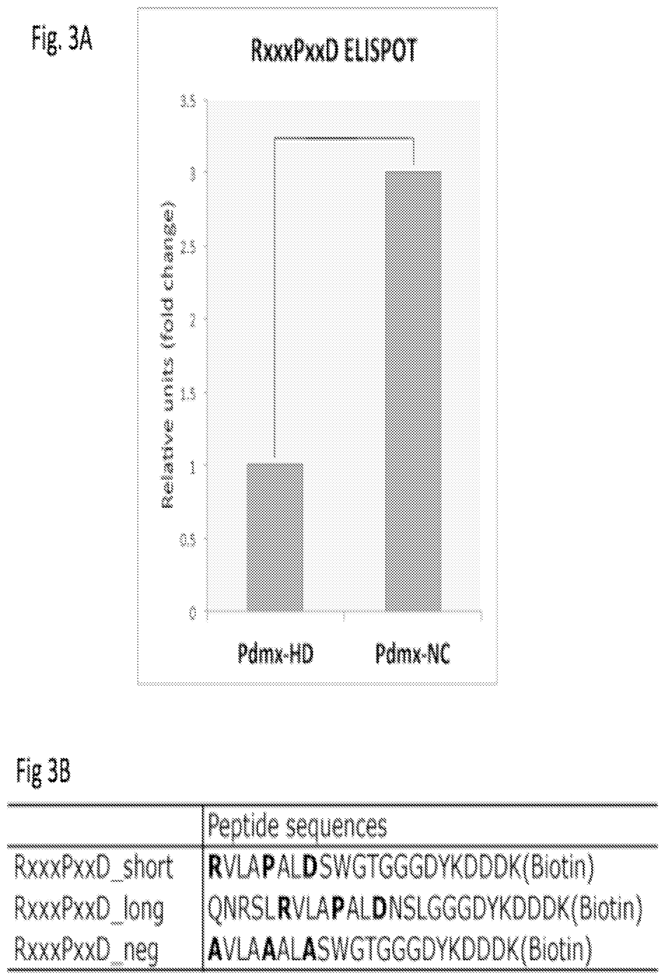

### DETAILED DESCRIPTION

Sequence listings:

SEQ ID NO: 1 is a narcolepsy-specific epitope.

SEQ ID NO: 2 is a preferable narcolepsy-specific epitope. Preferably, the residue in position 5 is P.

SEQ ID NO: 3 is a more preferable narcolepsy-specific epitope.

SEQ ID NO: 4 is a narcolepsy-specific epitope.

SEQ ID NO: 5 is a narcolepsy-specific epitope.

SEQ ID NO: 6 is a narcolepsy-specific epitope.

SEQ ID NO: 7 is a narcolepsy-specific epitope.

SEQ ID NO: 8 is a narcolepsy-specific epitope.

SEQ ID NO: 3 is a narcolepsy-specific epitope.

SEQ ID NO: 10 is a narcolepsy-specific epitope.

SEQ ID NO: 11 is a narcolepsy-specific epitope.

SEQ ID NO: 12 is a narcolepsy-specific epitope.

SEQ ID NO: 13 is a narcolepsy-specific epitope.

An epitope refers to a part of an antigen, such as a protein, that is recognized by the immune system, specifically by antibodies, B cells, or T cells. An epitope may be a linear epitope or a conformational epitope. The present disclosure describes epitopes as linear epitopes, the epitope optionally also comprising conformational epitopes that are recognized by the immune system similarly or identically as the corresponding linear epitope.

The amino acid sequences are described in this disclosure using the standard single letter code from the N-terminus to the C-terminus.

The term peptide refers herein to any compound comprising naturally occurring or synthetic amino acid polymers or amino acid-like molecules including but not limited to compounds comprising amino and/or imino molecules. Included within the definition are, for example, peptides containing one or more analogs of an amino acid (including, for example, unnatural amino acids, etc.), peptides with substituted linkages, as well as other modifications known in the art, both naturally occurring and non-naturally occurring (e.g., synthetic). Thus, synthetic oligopeptides, dimers, multimers (e.g., tandem repeats, linearly-linked peptides), cyclized, branched peptides and the like are included within the definition. Non-limiting lengths of peptides includes peptides having from 9 to 50 residues (or any integer between these extremes), from 10 to 40 residues, from 10 to 30 residues, or from 10 to 15 residues. Typically, peptides useful in this invention can have a maximum length suitable for the intended application. A person skilled in art can easily select the maximum length in view of the teachings herein. Further, peptides and polypeptides, as described herein, for example synthetic peptides, may include additional molecules such as labels or other chemical moieties. The peptides and polypeptides may also be artificially modified to include chemical bonds, substituents and/or atoms that are not present in peptides or polypeptides encountered in nature.

The reference composition may be positive or a negative control sample. A positive control sample may comprise as its components reagents giving a positive result in the test method, such as an antigen comprising a peptide of the seventh aspect or an antigen mimicking peptide as described above. A negative control sample may comprise similar or the same reagents as a positive control sample but without the component(s) required to give a positive result in the text. For example, if the test is carried out using serum samples, a positive sample may comprise serum or antibodies from a narcolepsy patient or an immunized host, such as a mouse or a rabbit. A negative control may comprise serum from a subject without narcolepsy.

The predetermined concentration level of an antibody to an antigen marker for narcolepsy may comprise a selected concentration or a titer level. Methods to determine such levels are known as such. A skilled person can determine an appropriate level to adjust the specificity and sensitivity according to needs.

In an embodiment the biological sample is a tissue sample derived from a subject. In another embodiment the biological sample is a biological fluid, such as blood, plasma, serum, urine, saliva, breast milk, or other serological sample.

In an embodiment of the eighth aspect the biological fluid is returned to the subject after removal of the antibodies. In another embodiment the biological fluid is collected and optionally stored after removal of the antibodies.

In an embodiment the subject is a mammal, such as an animal subject or a human subject, preferably a human subject. In an embodiment the subject has a population specific haplotype for narcolepsy. In a preferred embodiment the haplotype is HLA DQB1*06:02 allele.

In an embodiment the epitope specific for narcolepsy is present in a prostaglandin D2 receptor (also called DP1 and PTGDR1, Uniprot entry UQ13258).

In an embodiment the epitope specific for narcolepsy is an epitope of DP1.

In an embodiment the epitope specific for narcolepsy is present in a protein having at least 85% sequence identity with DP1, such as at least 85%, 86%, 87%, 88%, 89%, 90%, 91%, 92%, 93%, 94%, 95%, 96%, 97%, 98%, 99% or 100% with DP1. Preferably the protein comprises the sequence motif RxxxPxxD, such as RVLAPALD, SEQ ID NO: 3.

In an embodiment the epitope specific for narcolepsy is an epitope comprising the sequence of SEQ ID NO: 2, preferably comprising the sequence RxxxPxxD.

In an embodiment the epitope specific for narcolepsy is located at an extracellular loop of the protein, such as DP1 comprising the motif RxxxPxxD.

In an embodiment the epitope specific for narcolepsy is located at the 2^{nd} extracellular loop of the DP1 comprising the motif RxxxPxxD.

In another embodiment in step b. of the method is assayed the presence of an antibody which is specific for a further epitope of a protein, which protein also carries the epitope specific for narcolepsy as herein defined. In an embodiment the protein is DP1. In another embodiment the further epitope does not comprise the motif RxxxPxxD.

The inventors used different immunoanalysis methods and measured the serologic response in the studied sera to different peptides carrying the present epitope. The first confirmation of the association of the epitope with narcolepsy was by phage dot blot assay sowing that three out of four MVA-positive patient sera (Pdmx-NC1-3) showed distinct IgG reactivity at two different dilutions to the phage displaying peptide RVLAPALDSWGT. This target specificity was further confirmed by Western blot analysis using Pdmx-NC, where the interaction between human IgG and antigen expressing phage was blocked by the synthetic peptide RVLAPALDSWGT. By ELISA assay, serologic reactivity to RXXXPXXD peptide antigens was detected in Pdmx-NC. The inventors also found a wide range of DP1 mRNA and protein expression across human brain regions and in human cells used in further analysis. Finally, the detection of cellular DP1 by commercial anti-human-DP1 polyclonal sera were equal to detection using antibodies from human Pdmx-NC sera. Furthermore, synthetic peptide (RVLAPALDSWGT) was shown to compete for the same DP1 binding site as antibodies in sera of Pdmx-NC, but competition was not detected with control peptides or irrelevant sera, demonstrating DP1 as a novel true autoantibody target in narcolepsy.

In an embodiment the antibody specific for at least one epitope specific for narcolepsy is an autoantibody against DP1, or against an immunogenic part thereof.

In an embodiment wherein in step b. is assayed at least one further antibody preferably for narcolepsy; step c. comprises comparing the levels or ratios of said antibodies; and in step d. said subject is concluded to be at increased risk of having narcolepsy if the amounts of said antibodies for narcolepsy in said biological sample are higher than in the reference sample or are different by a determined concentration levels or ratios thereof. The further antibody may be an antibody specifically recognizing the narcolepsy-specific epitope according to the second aspect, an antibody specifically recognizing a further epitope in a protein comprising the narcolepsy-specific epitope according to the second aspect, or any other antigen marker for narcolepsy.

In an embodiment the method comprises assaying the biological sample for at least one further antibody specific for a pathogen carrying an antigen comprising the sequence of any one of SEQ ID NOs 1, 2 or 3 or their mimicking epitope; wherein the presence of the further antibody is indicative of an increased risk of narcolepsy. Such a pathogen epitope may be involved in the development of narcolepsy. An advantage of this embodiment may be an improved specificity or sensitivity of the test. A lower occurrence of false positives may also be achieved. The assay for the further antibody may be carried out before, after, or simultaneously with the step b. In an embodiment all antibody assays are carried out simultaneously.

In an embodiment the method comprises detecting the presence of a narcolepsy-specific haplotype, preferably HLA DQB1*06:02 haplotype, in the subject.

In another embodiment in the method the assaying is serological assay, preferably ELISA, ELISPOT, Enzyme Immunoassay Technique, RAST test, Radioimmunoassay, Radiobinding assay, Immunofluorescence, mimotope variation analysis (MVA), or a combination thereof.

In an embodiment in the method is for evaluating Pandremix-associated narcolepsy.

In an embodiment in the method is for evaluating sporadic narcolepsy.

In an embodiment in the method is for evaluating vaccine-associated narcolepsy.

In an embodiment in the method the epitope specific for narcolepsy is an epitope according to the second aspect.

In an embodiment of the method, the epitope specific for narcolepsy assayed in step b. is a further epitope present in a protein comprising the epitope according to the second aspect. This embodiment is advantageous in that it is allows using in detection another part, i.e. a further epitope, of the protein carrying the epitope according to the second aspect. Thus, it is not only the epitope of the second aspect which can be used in the detection, but another further epitope of the protein can be used as well, because the full-length protein contains the epitope of the second aspect.

In an embodiment the above protein is DP1.

In an embodiment the antibody is an immunoglobulin. In another embodiment the antibody is an autoantibody. In an embodiment the antibody is a synthetic antibody.

In an embodiment the epitope specific for narcolepsy comprises the sequence R-X-X-X-[P/A/V/L/I/P/W/F/M]-X-X-D, SEQ ID NO: 2.

In an embodiment the epitope specific for narcolepsy comprises the sequence R-V-L-A-P-A-L-D, SEQ ID NO: 3.

In an embodiment the epitope specific for narcolepsy is a further epitope present in a protein comprising the sequence R-X-X-X-[P/A/V/L/I/P/W/F/M]-X-X-D, SEQ ID NO: 2.

In an embodiment the epitope specific for narcolepsy is a further epitope present in a protein comprising the sequence R-V-L-A-P-A-L-D, SEQ ID NO: 3.

In an embodiment the biological sample is a serological sample.

In an embodiment of the eighth aspect the protein is an antibody or a B-cell receptor optionally bound to a cell.

Sample Collection and Manipulation

The particular anti-peptide serologic antibody profiles caused by narcolepsy can be identified on different biological samples derived form a tissues and body fluids. The biological sample is preferably selected from the group consisting of blood, urine, cerebrospinal fluid as well as from brain tissue. The sample may be used immediately for detection, or it can be sealed and transported to another location for analysis.

Detection

Different methods are available to detect narcolepsy-specific serologic response. Typical means include immunoassay methods, including ELISA, ELISPOT, Enzyme Immunoassay Technique, RAST test, Radioimmunoassay, Radiobinding assay, Immunofluorescence. These can be used as stand-alone detection or in different combinations.

Once the sample's serologic profile of biomarkers has been obtained, it is correlated with the diagnosis of narcolepsy. The determination of said levels of narcolepsy-specific biomarker can involve detecting whether said at least one biomarker is present or absent, or alternatively the levels in which it is present.

In an embodiment of the invention, the sample's serologic profile is compared with at least one reference negative profile acting as negative control sample, i.e. a serologic profile obtained from a subject or group of subjects known not have narcolepsy. In another embodiment the comparison is done with a positive reference serologic profile acting as positive control sample, i.e. a serologic profile obtained from a subject or group of subjects known to have narcolepsy. The positive control sample may have a predetermined concentration levels for the narcolepsy-specific protein biomarker. Thus, in order to determine the presence of narcolepsy the reference serologic profile can be positive or negative. The reference serologic can be obtained from a single subject or it can be obtained from a plurality of subjects.

According to one embodiment of the invention, the levels or abundance of antibodies to one antigen marker for narcolepsy are detected. In an alternative embodiment, the levels or abundance of antibodies to two or more antigen markers for narcolepsy are detected, and the determination of the biomarker profile of the sample can involve the detection of the levels or abundance and proportions in which different biomarker profiles are found.

### EXAMPLES

### Example 1

### Peptides Identified using MVA Analysis Discriminate Patients with and without Narcolepsy

Samples were analyzed using random peptide display (phage display) analysis (Brissette R, Goldstein N I., Methods Mol Biol. 2007; 383:203-13., Methods Mol Biol. 2007; 383:203-13). Shortly, 2 μl of human serum containing IgG (2 μl of normal human sera contains approximately 20 μg IgG) was used in affinity selection analysis. IgG was bound to capture agent—protein G coupled magnetic beads (New England BioLabs, S1430S) and incubated with Ph.D.™-12 Phage Display Peptide Library (New England BioLabs, E8111 L), thereafter IgG-phage complexes were separated by magnetic force, subjected to extensive washes, and direct phage lysis upon which DNA was amplified using insert-specific primers. Amplicons were sequenced and data of peptide frequency was analyzed. Only peptides reads equal to or >10 within analyzed samples were taken into bioinformatical analysis.

The following peptides according the presently identified sequence motif RxxxPxxD (a sequence according to the SEQ ID NO: 2) were identified by MVA from Pandremix-induced NC samples: RVLAPALDSLGT, RVLAPARDSWGT, SYRVEAPILDDS, RVLAPAHDSWGT, SLRVLEPVMDKK, RHLAPALDVWDL, RVMAPALDSWGT, RVLGPALDSWGT, LRVISPTLDSML, GRNLAPAQDTSI (SEQ ID NO:s 4-13, respectively). The results confirm that epitopes according to SEQ ID NO: 1, and SEQ ID NO: 2, are present in samples derived from subjects with narcolepsy. Said epitopes are specific for narcolepsy and can be used in detection assays for narcolepsy, in particular for Pandremix-associated narcolepsy.

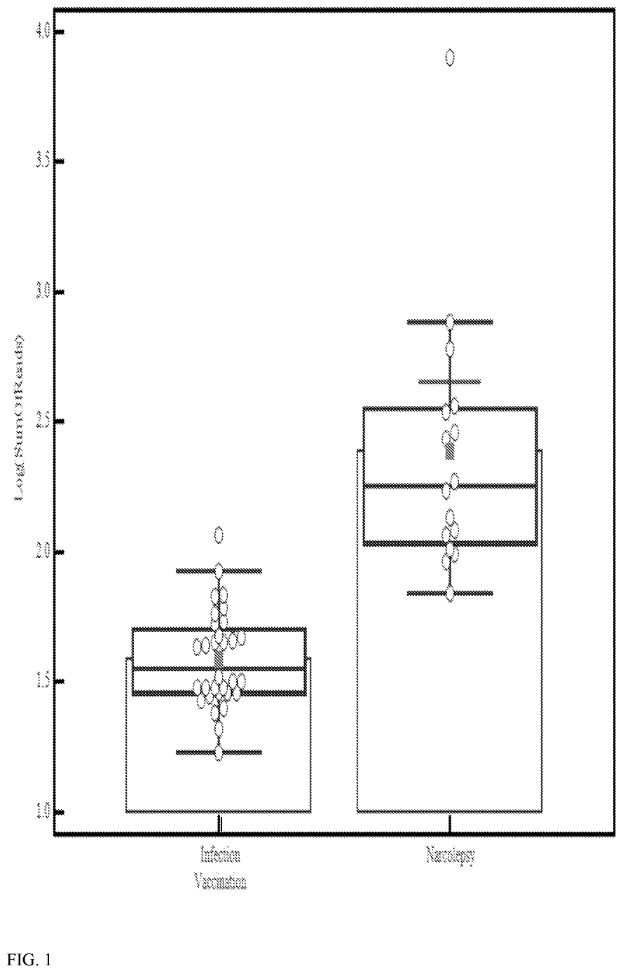

FIG. 1 shows results that seroreactivity against selected set of peptides distinguishing narcolepsy samples is significantly elevated in the narcolepsy group compared to the H1N1-infected and H1N1-vaccinated groups. Box-and-whisker plot showing Log-transformed values of total IgG response to Pandemrix-induced narcolepsy-specific peptide antigens (1644 peptides compiling the R...P..D motif) in samples from H1N1-infected and H1N1-vaccinated or from patients with narcolepsy. The logarithm-transformed IgG response values for the majority (i.e., the “box” part of the box-whisker plot) of narcolepsy samples (n=16) were greater than that of H1N1-infected and Pandemrix-vaccinated samples (n=16+16). The minimum, first quartile, median, third quartile, and maximum of the logarithm-transformed peptide antigen sum values were 1.25, 1.47, 1.66, 1.78.

Presented data clearly show that identified peptide epitopes can be used to discriminate narcolepsy patients from non/narcolepsy patients. This provides proof that clinical diagnostic tests to diagnose narcolepsy can be developed using these peptides, and that the peptides can be used to distinguish narcolepsy patients from individuals with no narcolepsy. It is a proof that the diagnostic peptides described can be used as claimed.

### Example 2

Methods

Spot ELISA

Human sera (NRC-S-0, NRC-S-59, NRC-S-25 and NRC-S-71 from University of Helsinki) were diluted 1:200 in the preabsorption solution containing mutated phages in 4% dry milk, and incubated over night at +4° C. on shaker. Phages (displaying peptide RVLAPALDSWGT) were diluted 1:2 and 1:4 in PBS containing 25% glycerol. 0.25 μl of phage dilution was dotted on nitrotcellulose membrane (Hybond_C Extra, Amersham Bioscience, Cat no. RPN 2020). Human IgG (50 ng/μl) was dotted as a positive control. After dotting, membranes were blocked with solution containing 4% dry milk in 1× PBS-Tween20 0.1% for 1 h at RT on an orbital shaker. Thereafter, the preabsorbed human sera were incubated with membrane for 1 h at RT on the shaker. For detection of bound IgG from human sera, Cy-5 conjugated Affinipure Goat anti-human IgG (H+L) (Jackson ImmunoResearch, 109-175-088) secondary antibodies 1:5000 were used. For normalization of the amount of phages across samples, mouse anti-M13 pVIII primary antibodies (GE, Healthcare, 27-9420-01) 1:5000 and Alexa Fluor 488 F(ab′)2 fragment of goat anti-mouse IgG (H+L) secondary antibodies (Invitrogen, Cat. no. A-11017) 1:5000 were used. Primary antibody incubation was done at RT for 45 minutes. Secondary antibody incubation was carried out in the dark at RT for 45 minutes. Results were scanned by using Ettan Digelmager (GE Healthcare Life Sciences).

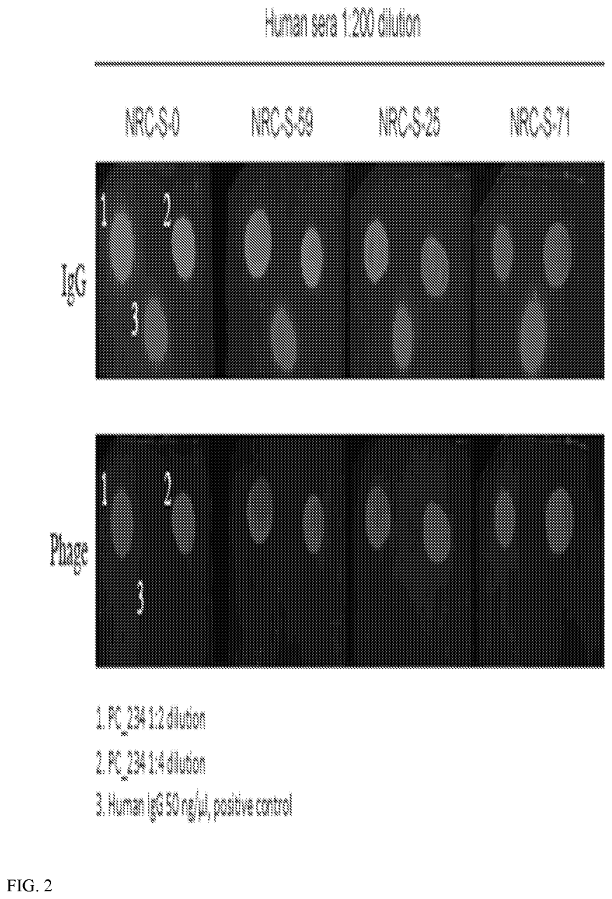

FIG. 2 illustrates evaluation of the predominant epitope carrying peptide with the RVLAPALDSWGT sequence for antibodies in the sera samples of diseased with narcolepsy. Evaluation of binding activity of screened RVLAPALDSWGT peptide by phage dot blot ELISA. Phages dotted at 2 different dilutions (1:2, 1:4) on nitrocellulose membrane were incubated with 4 different study sera diluted at 1:200 (NRC-S-0, NRC-S-59, NRC-S-25 and NRC-S-71). Upper part of the figure illustrates signal stemming of bound human IgG (visualized by Cy5-conjugated Affinipure Goat anti-Human IgG (H+L)) and lower part the staining of phages with mouse anti-M13 pVIII monoclonal antibody (visualized by Alexa fluor 488 F(ab′)₂ fragment of Goat anti-mouse IgG (H+L). Human IgG (50 ng/μl) was used as positive control (marked with number 3).

Presented data clearly show that using immunological methods (for example SPOT ELISA, plate ELISA, peptide array, lateral flow and flow/through based platforms), identified peptides can be used to develop immunological tests to diagnose narcolepsy. That enables development of fast, precise and inexpensive laboratory and Point of Care clinical diagnostic tools to diagnose narcolepsy.

1. A method of evaluating a subject for narcolepsy comprising
a. obtaining a biological sample from the subject;
b. assaying the biological sample for an antibody specific for at least one epitope specific for narcolepsy to obtain an assay result for the biological sample;
c. comparing the assay result for the biological sample with an assay result for the reference sample or with a predetermined concentration level of the antibody; and
d. concluding that said subject is at increased risk of having, or has acquired, narcolepsy if the assay result of the biological sample is higher than the assay result of said reference sample, or is different by a predetermined concentration level.

2. The method of claim 1 wherein in step b. is assayed at least one further antibody preferably for narcolepsy; step c. comprises comparing the levels or ratios of said antibodies; and in step d. said subject is concluded to be at increased risk of having narcolepsy if the amounts of said antibodies for narcolepsy in said biological sample are higher than in the reference sample or are different by determined concentration levels or ratios thereof.

3. The method of claim 1 comprising assaying the biological sample for at least one further antibody specific for a pathogen carrying an epitope comprising the sequence of any one of SEQ ID NOs 1, 2 or 3; wherein the presence of the further antibody is indicative of an increased risk of narcolepsy.

4. The method of claim 1, wherein the assaying is serological test, preferably ELISA, ELISPOT, Enzyme Immunoassay Technique, RAST test, Radioimmunoassay, Radiobinding assay, Immunofluorescence or a combination thereof.

5. The method of claim 1 for evaluating vaccine-associated narcolepsy or sporadic narcolepsy, preferably Pandemrix-associated narcolepsy.

6. The method of claim 1 wherein the epitope specific for narcolepsy comprises the sequence SEQ ID NO:1

7. The method of claim 1 wherein the epitope specific for narcolepsy assayed in step b. is a further epitope present in a protein comprising the sequence SEQ ID NO: 1.

8. The method of claim 1, wherein the epitope specific for narcolepsy assayed in step b. is a further epitope present in a protein comprising the sequence SEQ ID NO: 1, and wherein the protein is DP1.

9. An epitope specific for narcolepsy comprising the sequence SEQ ID NO: 1.

10. The epitope of claim 9 comprising the sequence SEQ ID NO: 2, preferably the sequence SEQ ID NO: 2 wherein the amino acid in position 5 is proline.

11. The epitope of claim 9 comprising the sequence SEQ ID NO: 3.

12. A glass, plastic, carbon, polymer-based, metallic or silicon plate, slide, chip or cartridge comprising epitope of claim 9 attached on it directly or by using a binding partner or a linker.

13. A kit comprising the glass, plastic, carbon, polymer-based, metallic or silicon plate, slide, chip or cartridge of claim 12, optionally a reference sample, and instructions for using it to assay a biological sample derived from a subject, preferably a human, to determine if said subject has or is at risk of developing narcolepsy.

14. A use of the epitope claim 9 as an immunoadsorbent for plasmapheresis or cytopheresis.

15. An antibody specific for the epitope of claim 9.

16. A peptide comprising the sequence SEQ ID NO: 1, SEQ ID NO: 2 or SEQ ID NO: 3, for treating narcolepsy.

17. A composition comprising a peptide comprising the sequence of SEQ ID NO: 1, SEQ ID NO: 2 or SEQ ID NO: 3, and optionally at least one of the following: a carrier, a buffer, and a preservative.

18. A method of targeted antibody removal from a biological fluid, preferably from plasma, of a subject in need thereof, the method comprising:
contacting a sample of biological fluid taken from a subject with a surface or particle to which is immobilized an epitope specific for narcolepsy, which selectively binds to a narcolepsy specific antibody suspected of being in the sample of biological fluid;
wherein the epitope comprises a peptide selected from SEQ ID NO: 1, SEQ ID NO: 2 and SEQ ID NO: 3.

19. An apheresis device comprising a peptide comprising a sequence selected from SEQ ID NO: 1, SEQ ID NO: 2 and SEQ ID NO: 3.


SEQUENCE LISTING (18 sequences)

SEQ ID NO: 1 - protein, 8 aa, Homo sapiens
  SITE (1)..(1): K, R, or H
  SITE (1)..(1): K, R, or H
  SITE (2)..(2): any residue
  SITE (3)..(3): any residue
  SITE (4)..(4): any residue
  SITE (5)..(5): A, V, L, I, P, W, F, or M
  SITE (6)..(6): any residue
  SITE (7)..(7): any residue
  SITE (8)..(8): D or E
XXXXXXXX

SEQ ID NO: 2 - protein, 8 aa, Homo sapiens
  SITE (2)..(2): any residue
  SITE (2)..(2): any residue
  SITE (3)..(3): any residue
  SITE (4)..(4): any residue
  SITE (5)..(5): A, V, L, I, P, W, F, or M
  SITE (6)..(6): any residue
  SITE (7)..(7): any residue
RXXXXXXD

SEQ ID NO: 3 - protein, 8 aa, Homo sapiens
RVLAPALD

SEQ ID NO: 4 - protein, 12 aa, Artificial sequence
  peptide
RVLAPALDSLGT

SEQ ID NO: 5 - protein, 12 aa, Artificial sequence
  peptide
RVLAPARDSWGT

SEQ ID NO: 6 - protein, 12 aa, Artificial sequence
  peptide
SYRVEAPILDDS

SEQ ID NO: 7 - protein, 12 aa, Artificial sequence
  peptide
RVLAPAHDSWGT

SEQ ID NO: 8 - protein, 12 aa, Artificial sequence
  peptide
SLRVLEPVMDKK

SEQ ID NO: 9 - protein, 12 aa, Artificial sequence
  peptide
RHLAPALDVWDL

SEQ ID NO: 10 - protein, 12 aa, Artificial sequence
  peptide
RVMAPALDSWGT

SEQ ID NO: 11 - protein, 12 aa, Artificial sequence
  peptide
RVLGPALDSWGT

SEQ ID NO: 12 - protein, 12 aa, Artificial sequence
  peptide
LRVISPTLDSML

SEQ ID NO: 13 - protein, 12 aa, Artificial sequence
  peptide
GRNLAPAQDTSI

SEQ ID NO: 14 - protein, 22 aa, Artificial sequence
  peptide
RVLAPALDSWGTGGGDYKDDDK

SEQ ID NO: 15 - protein, 26 aa, Artificial sequence
  peptide
QNRSLRVLAPALDNSLGGGDYKDDDK

SEQ ID NO: 16 - protein, 22 aa, Artificial sequence
  peptide
AVLAAALASWGTGGGDYKDDDK

SEQ ID NO: 17 - protein, 8 aa, Artificial sequence
  peptide
  SITE (2)..(4): Xaa can be any naturally occurring amino acid
  SITE (6)..(7): Xaa can be any naturally occurring amino acid
RXXXPXXD

SEQ ID NO: 18 - protein, 12 aa, Artificial sequence
  peptide
RVLAPALDSWGT